(19) United States
(12) Patent Application Publication
(10) Pub. No.: US 2024/0399055 A1
(43) Pub. Date: Dec. 5, 2024

(54) INJECTION DEVICE RECEIVING A SYRINGE

(71) Applicant: GUERBET, VILLEPINTE (FR)

(72) Inventors: Damien MATRAY, BOURGOIN-JALLIEU (FR); Jean DAVAL, VILLEURBANNE (FR); Barbara BLAIN, LYON (FR)

(73) Assignee: GUERBET, VILLEPINTE (FR)

(21) Appl. No.: 18/697,336

(22) PCT Filed: Sep. 30, 2022
(86) PCT No.: PCT/EP2022/077300

§ 371 Date: Mar. 29, 2024

(30) Foreign Application Priority Data
Sep. 30, 2021 (EP) 21306367.0

(51) Int. Cl. A61M 5/145

(52) U.S. Cl. CPC A61M 5/145

(57) ABSTRACT

An injection device (1) for delivering a medical fluid, comprising an interface module (4) configured to receive a syringe (6), the interface module (4) defining a reception space (40) adapted to receive a flange (12a) in the outer periphery of a barrel (10) of the syringe (6), a drive member (8) configured to couple a piston of the syringe, and to translate along a longitudinal axis (X). The interface module (4) comprises a lateral opening (46) defined between two supporting members (50) of the interface module (4) and extending radially in a first radial direction from the longitudinal axis (X), said lateral opening (46) adapted for a radial translation of the syringe (6) in the first radial direction to a loaded position in which the flange (12a) of the syringe (6) is received in the reception space and the barrel (10) of the syringe (6) extends along the longitudinal axis (X).

### TECHNICAL FIELD

The present invention generally relates to injection of medical fluids and, more particularly to an injection device and a syringe adapted to be received in the injection device to operate a piston of the syringe in order to inject a medical liquid.

### BACKGROUND OF THE INVENTION

Many medical procedures require injecting a medical fluid into a patient. For example, the injection of medical liquid such as iodinated contrast agent and saline solution is required in most CT scan diagnosis cases. Since such an injection requires a precise dose of injected medical fluid, this kind of injection is most often performed using an automated powered injection device. A syringe is placed in the injection device, and the movement of a drive member creates the vacuum or overpressure required to pump or inject a precise volume of medical fluid. An injection tubing connects the injector to the patient, thereby delivering the medical fluid to the patient.

A vast majority of injection devices include a drive member or plunger inserted into the syringe barrel and are based on a simple principle: the fluid contained into the syringe is pressurized by the application of a load on the rear face of the plunger. The plunger is mostly bonded and actuated by a motorized rod.

The syringe and the injection tubing form a disposable set, which is to be thrown away between each patient or when the medical fluid is changed, or simply periodically, to avoid any contamination. On the opposite, the powered injection device is reusable, intended to be used for several years with multiple patients and medical fluids. Changing the syringe of the injector is thus a routine procedure which must be optimized to be simple, easy to perform, without any risk of error for the operator. Since the piston is in contact with the medical fluid, the piston is part of the syringe.

Usually, the injection system has a two-level interface module between the injection device and the disposable set. A first interface module serves to attach the syringe body to the housing of the injection device and offers the mechanical stability required to pressurize the medical fluid within the syringe. The second interface module is between the piston and drive member of the injection device, to enable driving the piston along the syringe barrel to pressurize and inject the medical fluid.

Various syringe engagement and disengagement mechanisms are used in injection system. For example, based on a bayonet principle; the syringe is inserted into a reception space of the injection device by a translation along the longitudinal axis (axis of the motorized drive member), and the syringe is rotated to lock the syringe to the injection device. Such a mechanism is shown in U.S. Pat. No. 6,585,700B1 or patent application EP1767233A1. In utility model DE202004005433, the syringe is inserted into a reception space by a radial translation, then rotated. U.S. Pat. No. 9,308,316 discloses various structures which are used in a front-loading injector, for mounting the syringe to the injector and/or preventing leakage of spilled fluid from an external surface of a syringe into the vicinity of the drive ram.

In addition, the piston of the syringe needs to be connected to the drive member of the injection device. Some systems require that the piston be precisely positioned during the insertion of the syringe, the connection of the piston with the drive member being caused by the same movement as the insertion of the syringe or the locking of the syringe, as in utility model DE202004005433. Other systems rely on an actuator of the drive member to grab the syringe's piston, in order to allow pulling the piston and filling the syringe, as in patent applications WO2018/075379 and WO2019/168776.

Similarly, the removal of the syringe involves disconnecting the piston from the drive member before or simultaneously with the unlocking the syringe from the housing of the injection device. Once again, an actuator may be used, or a rotation, as in U.S. Pat. No. 6,585,700.

Patent application WO2019/046261 provides methods for calibrating the zero position of at least one drive member of an injector system is disclosed. Automated methods of position calibration of the drive member of a fluid injector are disclosed. These methods address sources of error in positional accuracy and fluid delivery inaccuracies, such as disposable syringe tolerance and injector wear over time. Methods and fluid injector systems for determining and correcting for the amount of slack in a fluid injection apparatus are described. An understanding of the calibration and the amount of slack in a fluid injection system allows a processor to correct for the slack, thereby ensuring more accurate fluid delivery to the patient and more accurate imaging processes.

Patent application US2018/304016 discloses A device for rapidly loading and releasing a syringe. A limiting flange and clamping flange are on an outer side wall of an end, connected to the device, of the syringe. The clamping flange is close to a rear end of the syringe. The device includes an injection head. An outer collar, middle plate, and inner collar are sequentially inside the injection head. The outer collar includes an upper and lower part that are disposed up and down. The inner collar includes an upper part of the inner collar and a lower part of the inner collar that are disposed up and down. Elastic structures are between the upper part of the inner collar and injection head and between the lower part of the inner collar and injection head. The injector syringe can be loaded onto the device and taken out rapidly if the syringe is rotated.

Patent application WO02/056945 discloses a syringe interface or adapter includes a first retaining member that pivots about an axis generally perpendicular to and offset from the axis of an injector piston when the syringe interface is attached to the injector. The first retaining member pivots in a rearward direction to engage a portion of the syringe flange. The syringe interface further includes a second retaining member slideably positioned within a passage in the syringe interface to slide in a direction generally perpendicular to the axis of the piston when the syringe interface is attached to the injector to engage a portion of the syringe flange.

The existing systems show several limitations. First, most systems require the syringe to be properly positioned to allow the insertion of the syringe into the injection device. For example, systems based on a bayonet principle require that the syringe be inserted at a precise angular position, which complicates the preparation of the insertion. In addition, many systems involve a rotation of the syringe, which requires a complex handling system or the intervention of an operator manually rotating the syringe. This also prevents connecting several syringes to an injection device with several drive members in a single step.

Since the drive member is meant to move within the syringe along a longitudinal axis, most systems require an imposed positioning of the drive member, most often in a retracted position, to be able to insert or remove the syringe. The same goes for connecting the syringe's piston to the drive member: the need for a precise positioning of the drive member or the piston entails the need for measuring their respective positions, or may lead to problems if they are not in the requisite positions.

The use of an actuator or of an active sub-system for connecting or disconnecting the piston and the drive member adds complexity to the injection device, and increases costs and risks of malfunctions occurring.

Accordingly, there is a need for an injection device able to receive a syringe and connect a drive member to the syringe's piston in a single step, in a simple and easy way, allowing also an easy removal of the syringe, regardless of the position of the drive member, without any active part.

### SUMMARY OF THE INVENTION

It is proposed an injection device for delivering a medical fluid, comprising:
an interface module configured to receive a syringe, the interface module defining a reception space adapted to receive a flange in the outer periphery of a barrel of the syringe,
a drive member configured to couple a piston of the syringe, wherein said drive member is configured to translate along a longitudinal axis through the reception space,
wherein the interface module comprises a lateral opening defined between two supporting members of the interface module and extending radially in a first radial direction from the longitudinal axis, said lateral opening being adapted for a radial translation of the syringe in the first radial direction to a loaded position in which the flange of the syringe is received in the reception space and the barrel of the syringe extends along the longitudinal axis, and wherein each supporting member comprises a folding portion pivotable between a supporting position in which said folding portion forms a supporting surface for the flange of the syringe, and an open position in which said folding portion opens a passage parallel to the longitudinal axis for the flange of the syringe, thereby allowing a longitudinal translation of the syringe out of the loaded position.

Other preferred, although non-limitative, aspects of the invention are as follows, isolated or in a technically feasible combination:
the folding portion of the supporting member is radially offset with respect to the longitudinal axis, in the radial direction of the lateral opening;
the supporting surface formed by the folding portion extends in a second radial direction, opposite the first radial direction, beyond the reception space towards the lateral opening;
the folding portion of a supporting member is formed by a hinged door pivotable perpendicularly with respect to the longitudinal axis between the supporting position, and the open position;
the hinged door is biased into the supporting position by a biasing member;
the drive member comprises a head provided with a first coupling organ configured to couple to a second coupling organ of the piston when said first coupling organ is pressed on said second coupling organ, wherein the first coupling organ is configured to retain said second coupling organ along the longitudinal axis without retaining said second coupling organ in a second radial direction opposite the first radial direction;
the interface module comprises a pair of jaws flanking the reception space, the jaws being pivotable between a clamping position in which the jaws are closed in the reception space, and an open configuration in which the jaws are open, wherein said pair of jaws are maintained in the clamping configuration by a biasing force, and wherein said pair of jaws are maintained in the open configuration by a biasing force.

It is also proposed an injection device for delivering a medical fluid, comprising:
an interface module configured to receive a syringe, the interface module defining a reception space adapted to receive a flange in the outer periphery of a barrel of the syringe,
a drive member configured to couple a piston of the syringe, wherein said drive member is configured to translate along a longitudinal axis through the reception space,
wherein the interface module comprises a lateral opening defined between two supporting members of the interface module and extending radially in a first radial direction from the longitudinal axis, said lateral opening being adapted for a radial translation of the syringe in the first radial direction to a loaded position in which the flange of the syringe is received in the reception space and the barrel of the syringe extends along the longitudinal axis, and wherein the drive member comprises a head provided with a first coupling organ configured to couple to a second coupling organ of the piston when said first coupling organ is pressed on said second coupling organ as the drive member translates along the longitudinal axis into the inner volume of the barrel, wherein the first coupling organ is configured to retain said second coupling organ along the longitudinal axis without retaining said second coupling organ in a second radial direction opposite the first radial direction.

The interface module may comprise a pair of jaws flanking the reception space, the jaws being pivotable between a clamping position in which the jaws are closed in the reception space, and an open configuration in which the jaws are open, wherein said pair of jaws are maintained in the clamping configuration by a biasing force, and wherein said pair of jaws are maintained in the open configuration by a biasing force.

The invention also relates to a syringe assembly comprising a plurality of syringes mounted on a same holder, wherein each syringe comprises a barrel extending along a longitudinal direction and defining an inner volume extending between a first end and a second end, wherein the first end is open and allows a drive member of an injection device to translate along a longitudinal axis within the barrel, each syringe comprising a piston within the barrel, wherein the barrel is provided with at least a flange in the outer periphery of the barrel, and wherein the piston comprises a coupling organ configured to cooperate with the drive member of an injection device, said coupling organ being a projecting part comprising a head and a neck, said head larger than the neck in a radial direction, said neck connecting the head to the rest of the piston.

Other preferred, although non-limitative, aspects of the syringe assembly are as follows, isolated or in a technically feasible combination:
the holder is provided with a plurality of brackets for holding a respective syringe, wherein the syringes are aligned;
the barrel of each syringe is provided with at least a flange in the outer periphery of the barrel, said flange being configured to be received in a reception space of an interface module of an injection device in accordance with the invention in a configuration in which the barrel extends along the longitudinal axis.
the barrel of each syringe is provided with an upper flange and a lower flange in the outer periphery of the barrel, said two flanges distributed along the longitudinal direction, the upper flange closer to the open first end of the barrel than the lower flange.

The invention also relates to an injection system comprising the injection device of the invention and the syringe assembly of the invention.

The invention also relates to an unloading process for removing a syringe from an injection device in an injection system in accordance with the invention, wherein each supporting member comprises a folding portion pivotable between a supporting position in which said folding portion forms a supporting surface for the flange of the syringe, and an open position in which said folding portion opens a passage parallel to the longitudinal axis for the flange of the syringe, thereby allowing a longitudinal translation of the syringe out of the loaded position, said unloading process comprising two steps:
a first step of radially translating the syringe in a second radial direction, opposite the first radial direction, from the reception space toward the lateral opening of the interface module, so that a flange of said syringe faces a folding portion of a supporting member, and
a second step of longitudinally translating the syringe in a third, longitudinal, direction parallel to the longitudinal axis, thereby causing the folding portion of the supporting member to fold and allow a longitudinal translation of the syringe out of the loaded position.

The invention also relates to an unloading process for removing a syringe from an injection device in an injection system in accordance with the invention, wherein the drive member comprises a head provided with a first coupling organ configured to couple to a second coupling organ of the piston when said first coupling organ is pressed on said second coupling organ, wherein the first coupling organ is configured to retain said second coupling organ along the longitudinal axis without retaining said second coupling organ in a second radial direction opposite the first radial direction, said unloading process comprising three steps:
a first step of radially translating the syringe in a second radial direction, opposite the first radial direction, from the reception space toward the lateral opening of the interface module, thereby disengaging the first coupling organ and the second coupling organ,
a second step of removing the drive member from an inner volume of the syringe,
a third step of radially translating the syringe in the second radial direction out of the lateral opening of the interface module.

The invention allows inserting and removing a syringe from an injection device without requiring any rotation of the syringe, thereby allowing several syringes mounted on a same holder to be handled together.

### BRIEF DESCRIPTION OF THE DRAWINGS

Other aspects, objects and advantages of the present invention will become better apparent upon reading the following detailed description of preferred embodiments thereof, given as non-limiting examples, and made with reference to the appended drawings wherein:

FIG. 1a is a front view of an injection system in accordance with possible embodiments of the invention;

FIG. 1b is a cross-sectional view of the injection system of FIG. 1a;

FIG. 2 is a perspective view showing an interface module and a syringe in accordance with a possible embodiment of the invention, with the syringe not yet loaded into the interface module;

FIG. 3a is a perspective view showing an interface module and a syringe assembly in accordance with a possible embodiment of the invention, with the syringes held together by a holder and are not yet loaded into the interface module;

FIG. 3b is a cross-sectional view of a syringe mounted on a holder in a syringe assembly

FIG. 4a is a perspective view showing an example of a holder in an expanded configuration and in a contracted configuration, in accordance with possible embodiments of the invention;

FIG. 4b is a perspective view showing an example of a syringe assembly in an expanded configuration, in accordance with possible embodiments of the invention;

FIG. 5 is a perspective view showing an interface module showing components of the folding portions of the supporting members, in accordance with possible embodiments of the invention;

FIG. 6 is a front view showing the lateral opening and the reception space of an interface module, in accordance with possible embodiments of the invention;

FIG. 7 is an exploded view of an interface module showing different components of said interface module, in accordance with possible embodiments of the invention;

FIGS. 8a-8e are enlarged, cross-sectional, top views showing how the jaws act when the syringe is inserted into the reception space, in accordance with possible embodiments of the invention;

FIGS. 9a-9c are cross-sectional views showing the coupling between the drive member and the piston head, in accordance with possible embodiments of the invention;

FIGS. 10a-10e illustrate, with perspective and cross-sectional views, the arrangement of components of the injection system when the syringe is loaded in the reception space of the interface module, in accordance with possible embodiments of the invention;

FIGS. 11a-11d illustrate, with perspective and cross-sectional views, the arrangement of components of the injection system during a first stage of removal of a syringe from the interface module, in accordance with possible embodiments of the invention;

FIGS. 12a-12d illustrate, with perspective and cross-sectional views, the arrangement of components of the injection system during a second stage of removal of a syringe from the interface module, in accordance with possible embodiments of the invention;

FIG. 13a is a front view of an injection system in accordance with possible embodiments of the invention, without folding portion;

FIG. 13b is a cross-sectional view of the injection system of FIG. 13a;

FIG. 14 is a perspective view showing an interface module and a syringe in accordance with a possible embodiment of the invention, with the syringe not yet loaded into the interface module;

FIG. 15 is an exploded view of an interface module showing different components of said interface module, in accordance with possible embodiments of the invention;

FIGS. 16a-16e illustrate, with perspective and cross-sectional views, the arrangement of components of the injection system when the syringe is loaded in the reception space of the interface module, in accordance with possible embodiments of the invention;

FIGS. 17a-17d illustrate, with perspective and cross-sectional views, the arrangement of components of the injection system during a removal of a syringe from the interface module, in accordance with possible embodiments of the invention.

### DETAILED DESCRIPTION OF THE INVENTION

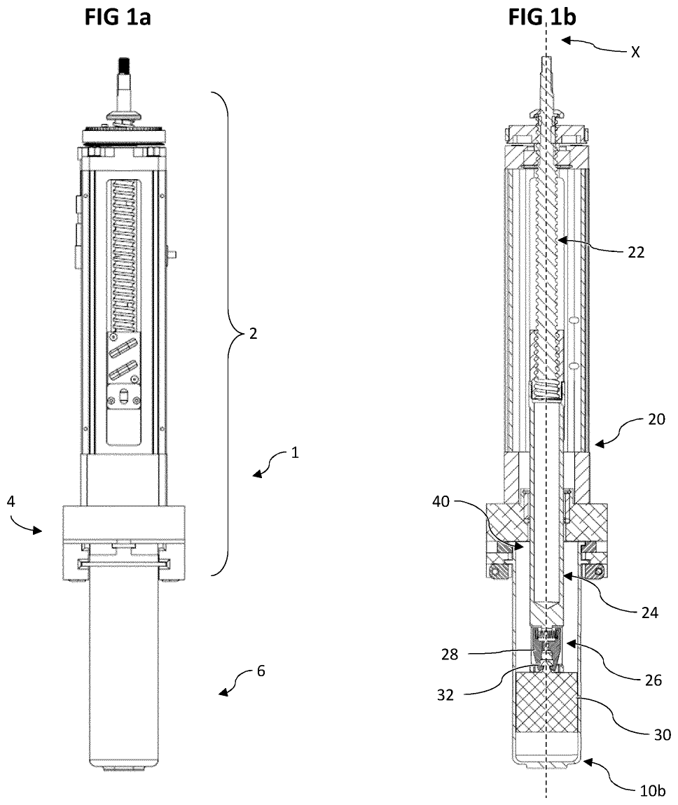

FIG. 1a shows a view of an injection system 1, and FIG. 1b shows a cross-section of the injection system 1. The injection system 1 includes an injection device 2 including an interface module 4 in which is loaded a syringe 6, in the mounted configuration wherein the injection system 1 can be used. Such an injection system 1 is used to inject any medical fluid into a patient, such as contrast agent. The medical fluid is provided from a medical fluid container (not shown) coupled to the syringe 6. Typically, the injection device 2 is sized and configured to accommodate use for multiple injections (e.g., multiple injections of one, but more typically multiple patients) and with different medical fluids, whereas the syringe 6 is configured to be a single-use device, for example for one type of medical fluid or for one patient only, one for a limited duration of time (daily set). The injection device 2 includes a drive member 8 configured to translate along a longitudinal axis, thereby defining said longitudinal axis. A radial direction is a direction extending in a plane perpendicular to the longitudinal axis and intersecting said longitudinal axis.

In most of the figures such as FIGS. 1a and 1b, the injection system 1 is represented in a vertical configuration, i.e. with the longitudinal axis aligned with the vertical, with the injection device 2 above the syringe 6. There is however no such positioning requirement, and the injection system could be rotated in an oblique or horizontal configuration. However, for the sake of simplicity and clarity, the following description may refer to relative spatial indications such as “upwards”, “downwards”, “above”, “below”, etc., which must be construed as non-limiting and used to describe the appended drawings.

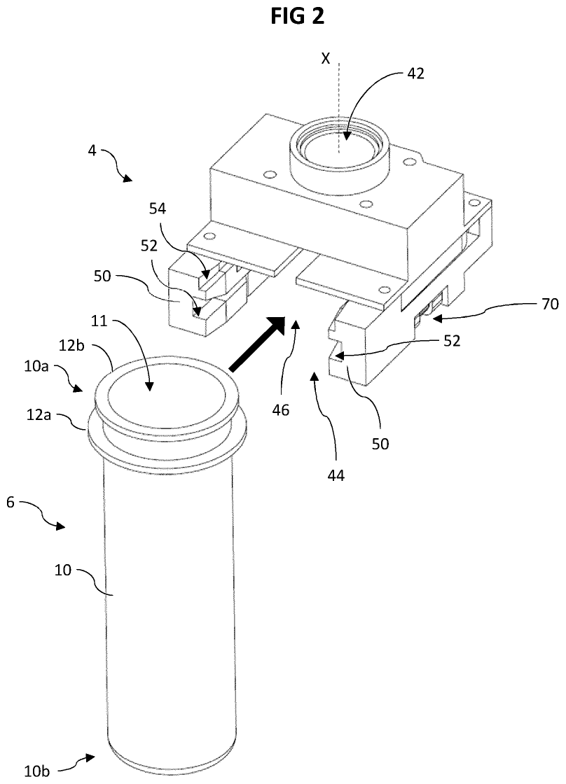

FIG. 2 more clearly shows the syringe 6 and the interface module 4. The syringe 6 includes a barrel 10, preferably a cylindrical hollow body, with a first end 10a and a second end 10b, which defines an inner volume 11 for the syringe 6. The inner volume 11 extends between the first end 10a and the second end 10b. The first end 10a is open and allows a drive member 8 of the injection device 2 to translate along the longitudinal axis within the barrel 10 of the syringe 6. The second end 10b includes a tubing interface adapted for connecting a tubing to the syringe 6 for transporting the medical fluid into and out of the syringe 6. The syringe 6 is provided with a piston 30 located within the inner volume 11 defined by the barrel 10. Preferably, the syringe 6 is symmetrical with respect to the longitudinal direction.

The barrel 10 is provided with at least a flange 12a in the outer periphery of the barrel 10. The flange 12a continuously encircles the barrel 10, and is perpendicular to the longitudinal axis X. The flange 12a has a circular shape. Preferably, as depicted in the figures, the barrel 10 is provided with two flanges 12a, 12b: an upper flange 12b and a lower flange 12a, the upper flange 12b closer to the open first end 10a of the barrel 10 than the lower flange 12a. Both flanges 12a, 12b are however close to each other, at the same side of the barrel 5 near the first end 10a and at distance from the second end 10b. The flanges are preferably similar in shape. A flange may form the extremity of the barrel 10 at the first end 10a thereof, as the upper flange 12b in the depicted example. In one aspect of the present disclosure, the lower flange 12a has a larger outer diameter than the upper flange 12b to facilitate downward extraction.

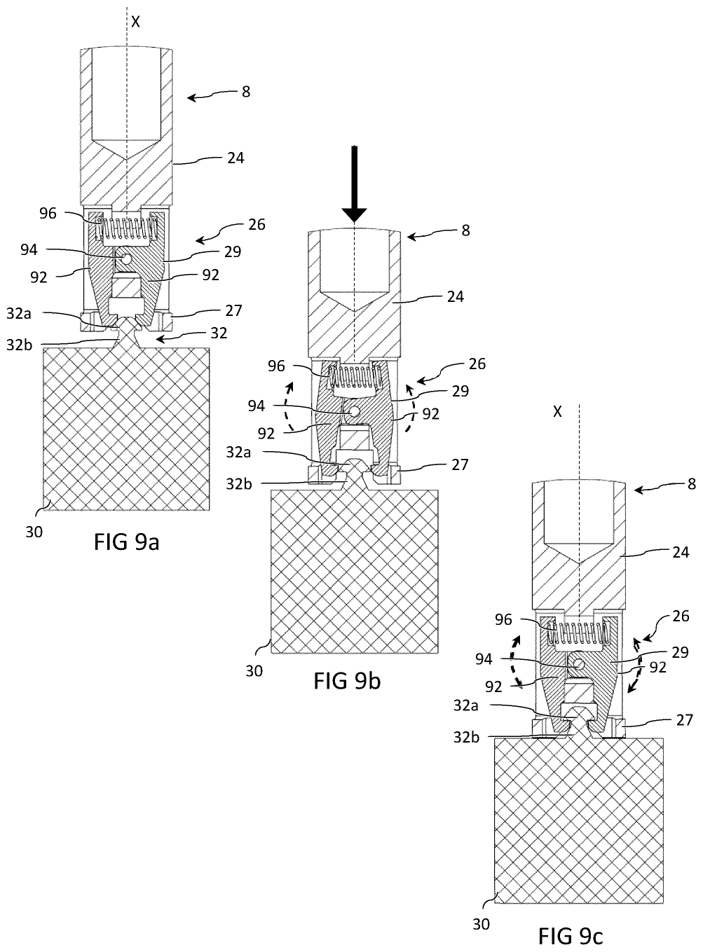

The injection device 2 includes an interface module 4 configured to receive the syringe 6, and a drive member 8 (illustrated in FIGS. 9a-9c). The drive member 8 is configured to translate along a longitudinal axis through the interface module 4 and within the barrel 10 of the syringe 6. The injection device comprises an actuator 20 for driving the drive member 8 into translation. The actuator 20 is typically a linear actuator, and most notably an electric actuator including an electric motor mechanically connected to rotate a lead screw 22 on which is mounted a mobile shaft 24 which is translated along the longitudinal axis X, and forms the part of the drive member 8 configured to traverse the interface module to translate within the barrel 10. Other types of actuators may be used. The drive member 8 is configured to couple with a piston 30 of the syringe 6. More precisely, the drive member 8 is terminated by a head 26 provided with a first coupling organ 28 configured to couple to a second coupling organ 32 of the syringe's piston 30 when said first coupling organ 28 is pressed on said second coupling organ 32.

The interface module 4 forms an interface between the drive member 8 and the syringe 6. The interface module 4 defines a reception space 40 adapted to receive and accommodate the flange 12b of a syringe 6, with a syringe's barrel 10 extending along the longitudinal axis X. In FIGS. 1a and 1b, the syringe 6 is loaded in the injection device 2 and therefore the first end 10a of the barrel 10 is received in the reception space 40 of the interface module 4 of the injection device 2. More precisely, the two flanges 12a, 12b are inserted into the interface module 4 and are accommodated in the reception space 40.

The interface module 4 also includes a hole 42 along the longitudinal axis X to allow the drive member 8 to penetrate into the reception space 40. In the depicted example, the through hole is provided with connection means such as a thread so that a housing of the actuator 20 can be fixed to the interface module 4. The interface module 4 has an open side 44 opposite the hole 42, in communication with the reception space 40, to allow the syringe 6 to extend along the longitudinal axis X when the syringe 6 is loaded in the first interface module. The interface module 4 includes a lateral opening 46 in communication with the reception space 40 and adapted for a radial translation of the syringe 66 in a first, radial, direction to a loaded position in which the flange 12a of the syringe 6 is received in the reception space 40 and the barrel 10 of the syringe 6 extends along the longitudinal axis X, thanks to the open side 44 of the interface module 4. The lateral opening 46 thus extends in a radial direction from the longitudinal axis X, so that the reception space 40 is laterally opened by said lateral opening 46.

The lateral opening 46 is defined between two supporting members 50. Each supporting member 50 has a guiding surface 52 extending in the radial direction to guide the flange 12a of the syringe during the insertion of the syringe. Preferably, as shown, each supporting member 50 has a guiding surface 52, 54 for each of the two flanges 12a, 12b: a lower guiding surface 52 for the lower flange 12a and an upper guiding surface 54 for the upper flange 12b. As shown in this example, a guiding surface 52 can be formed by a groove or slit arranged in a supporting member 50. Each supporting member 50 also has a supporting surface 53 extending in the radial direction to maintain and support a flange 12a in the longitudinal direction.

FIG. 2 shows a single syringe 6 about to be loaded into an interface module 4 with a single lateral opening 46 and a single reception space 40. A fat arrow shows the first radial direction for the radial translation of the syringe 6 required for loading syringe 6 in the interface module 4, until a loaded position in which the flange 12a of the syringe 6 is accommodated in the reception space 40 and the barrel 10 of the syringe 6 extends along the longitudinal axis X.

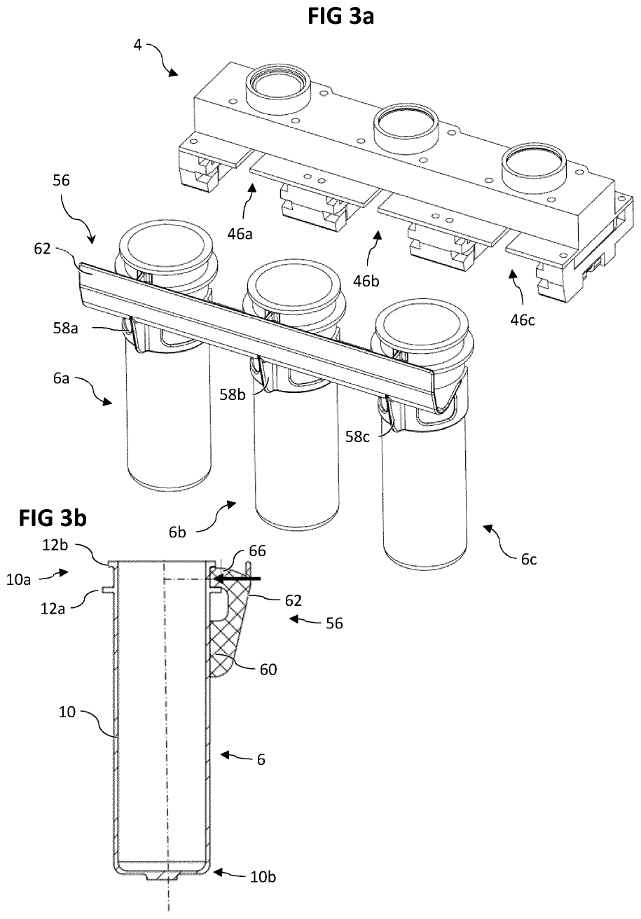

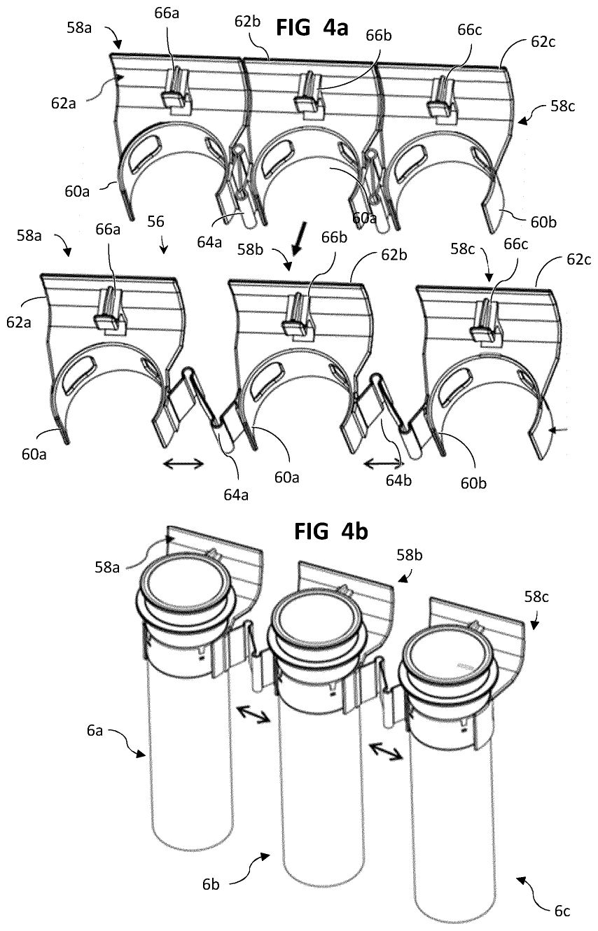

The configuration of the injection system 1 advantageously allows several syringes 6a, 6b, 6c to be loaded simultaneously by a same translation into several interface modules 4 or into an interface module 4 provided with several reception spaces 40 and corresponding lateral openings 46a, 46b, 46c, as illustrated in FIG. 3a. The syringes 6a, 6b, 6c are assembled in a syringe assembly. Preferably, the syringes 6a, 6b, 6c of a syringe assembly are mounted on a holder 56 provided with a plurality of brackets 58a, 58b, 58c for holding a respective syringe 6a, 6b, 6c, as shown in FIGS. 3a, 4a and 4a. Typically, the syringes 6a, 6b, 6c mounted on the holder 56 are parallel and aligned in a direction which is perpendicular to the longitudinal direction for the purpose of their insertions in the lateral openings 46a, 46b, 46c. Similarly, the plurality of reception spaces 40 of the interface module 4 are aligned in a direction perpendicular to the first radial direction, with their corresponding lateral openings 46a, 46b, 46c extend in the first radial directions.

In the examples of FIGS. 3a and 4a-4b, the holder 56 is configured to receive three syringes 6. However, a syringe assembly may comprise a various number of syringes 6, just by providing the holder 56 with the corresponding number of brackets 58. Even though the syringe assembly preferably comprises at least two or more syringes 6, a syringe assembly may comprise only one syringe 6 mounted on a holder 56 provided with a unique bracket 58. Each bracket 58 is configured to support one syringe 6, and more precisely is configured to cooperate with the barrel 10 of the syringe 6 supported by said bracket 58.

In the depicted embodiment, each bracket 58a, 58b, 58c includes a partial collar 60a, 60b, 60c configured to receive and retain a syringe 6, and more precisely the barrel 10 of a syringe 6. Each partial collar 60a, 60b, 60c is coupled to a support frame 62, 62a, 62b, 62c. The support frame 62, 62a, 62b, 62c may be common to several brackets 58a, 58b, 58c, as in FIG. 3a, or may be specific to each bracket 58a, 58b, 58c, as in FIG. 4a. In this case, the brackets 58a, 58b, 58c may be linked together by a mechanical connection 64a, 64b, which may be foldable to modify the spacing of the brackets 60a, 60b, 60c. This embodiment allows packaging the holder 56 in a retracted or folded configuration to save volume, and to adapt the spacing of the brackets 58a, 58c, 58c to reach the desired spacing corresponding to the distance between two lateral openings 46a, 46b, 46c of the interface module 4 in an expanded configuration. To each partial collar 60a, 60b, 60c may be associated a flange spacer 66a, 66b, 66c projecting from the support frame 62a, 62b, 62c above said bracket 58a, 58b, 58c so that the flange spacer 66a, 66b, 66c contacts the barrel 10 below a flange 12b, and preferably between the two flanges 12a, 12b of the barrel.

As more visible in FIG. 3b, the partial collar 60 embraces the barrel 10 below the lower flange 10a. A syringe 6 is mounted on the holder 56 simply by pushing the barrel 10 of the syringe 6 into the open portion of a partial collar 60. To this end, the partial collar 60 is configured to be elastically deformed upon the insertion of a syringe 6 to allow the barrel 10 to penetrate within the partial collar 60, and to retain it. The flange spacer 66 is introduced between the two flanges 10a, 10b by applying a force on the flange spacer 66, as illustrated by the arrow. The flange spacer 66 then contacts the barrel 10. The flange spacer 66 is adapted to the spacing between the two flanges 12a, 12b so that it is wedged between the two flanges 12a, 12b and is thus held in place.

Preferably, the syringes 6 are kept on the holder 56, forming a syringe assembly, throughout the operation of the injection device 1. This means that the syringes 6 are kept assembled from the loading process, in which the syringes 6 are inserted in the injection device 1, to the unloading process for removing the syringes from the injection device 1. For the sake of clarity and simplicity, in the other Figures, only one syringe 6 is represented, without the holder 56 holding said syringe 6, even if it may be present. Alternatively, it would be possible to remove the holder 56 once the syringes are inserted, and to bring back the holder 56 to remove the syringes 6. In either case, the syringes 6 are mounted on the holder 56 during the insertion process and the removal process.

Using such a syringe assembly is possible because the injection device 1 is configured to allow the insertion and the removal of the syringes 6 without requiring any rotation of the syringes 6. The insertion and the removal of the syringes 6 is made only with translations of the syringe assembly.

Each supporting member 50 of the interface module 4 comprises a folding portion 70 pivotable between a supporting position in which said folding portion 70 forms a supporting surface 53 for the flange 12a of the syringe 6, and an open position in which said folding portion 70 opens a passage parallel to the longitudinal axis X for the flange 12a of the syringe, thereby allowing a longitudinal translation of the syringe 6 out of the loaded position. In the supporting position, the supporting surface 53 continues or prolongs the guiding surface 52 of the supporting member 50 in the reception space 40, so that during the insertion of the syringe 6 by a radial translation in the first radial direction, a flange 12a of the syringe 6 is guided first by the guiding surface 52, and then is guided and supported by the supporting surface 53 of the folding portion 70 once said syringe is in the reception space 40.

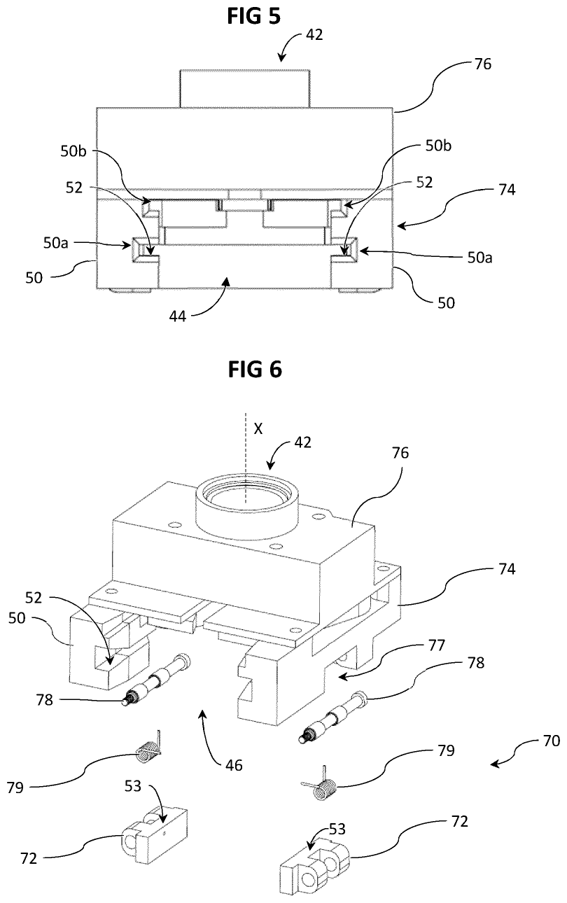

FIGS. 5, 6 and 7 show details of the interface module 4 in an exemplary embodiment. FIG. 5 shows a front view of the assembled interface module 4, facing the lateral opening 46, while FIG. 6 is a view of the interface module 4, in which the hinged doors 72 forming the folding portions 70 are detached. The interface module 4 includes a main part 74 on which is fixed a cover 76. The cover hole 42 goes through the cover 76 and ensures the centering and guidance of the drive member 8 passing through with respect to the interface module 4. The cover hole therefore preferably has a section corresponding to the section of the drive member. The cover 76 may be fixed on the main part 74 by fastening organs such as screws passing through fixation holes provided in the cover 76 and main part 74.

The main part 74 of the interface module has one supporting member 50, or arm, on either side of the lateral opening 46. The two supporting members 50 define two respective guiding surfaces 52 for a syringe's flange 12a. Preferably, each of the supporting members 50 has a groove 50a, a lateral surface thereof being the guiding surface 52. The grooves 50a are open on the side of the lateral opening 46 opposite the reception space 40. A wall of a groove 50a is formed by the guiding surface 52 of the supporting member 50 then by the supporting surface 53 of the supporting member 50, which is a surface of the folding portion 70. Since the syringe has preferably two flanges 12a, 12b, the interface module 4 preferably has a groove 50a for guiding and supporting the lower flange 12a and a slot 50b for guiding and supporting the lower flange 12b.

Each supporting member 50 bears a hinged door 72 forming a folding portion 70 for said supporting member 50. A supporting member 50 therefore includes an arm of the main part 74 and the hinged door 72 mounted on said main part 74. The hinged door 72 is pivotable perpendicularly with respect to the longitudinal axis X between a support position in which the hinged door 72 forms the supporting surface 53 for the flange 12a of the syringe 6, and a release position in which the hinged door 72 opens a passage through the open side 44 of the interface module 4, thereby allowing a longitudinal translation of the syringe 6 out of the reception space.

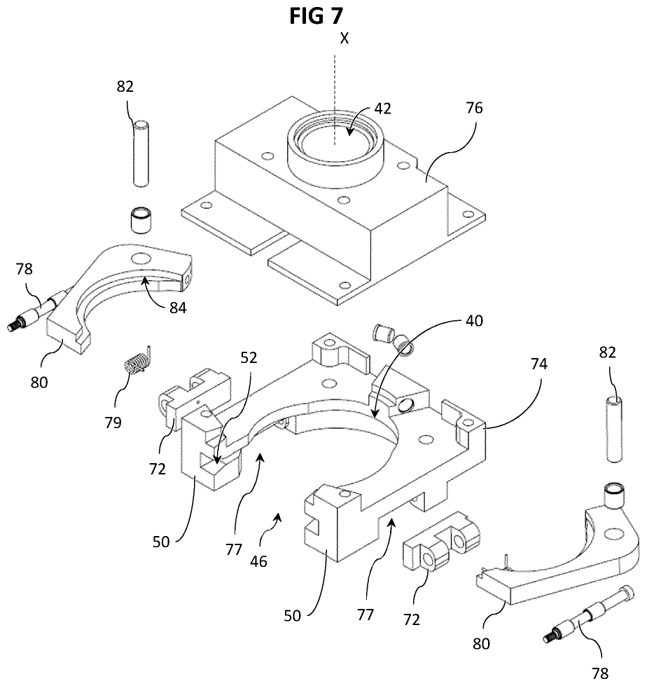

In this example, a hinged door 72 is supported on the main part 74 by a pin 78 passing by knuckles of the hinged door 72. The hinged door 72 is biased into the support position by a biasing member 79, such as a torsion spring like the illustrated helical torsion springs. In FIGS. 6 and 7, because of the removal of the hinged doors 72 of the supporting members 50, a void 77 is left after the guiding surface 52.

Preferably, the folding portion 70 of the supporting member 50 is radially offset in the radial direction of the lateral opening 46 with respect to the longitudinal axis X. The supporting surface 53 formed by the folding portion 70 of the supporting member 50 may thus not be centered in the reception space 40, but a part of the supporting surface 53 formed by the folding portion 70 extends in a second radial direction, opposite the first radial direction, beyond the reception space 40 towards the lateral opening 46.

Typically, the supporting surface 53 formed by the folding portion 70 of the supporting member 50 extends over the whole length of the reception space 40, so that when the syringe 6 is loaded into the injection device 2, the flange 12a of the syringe 6 is received in the reception space 40 and is entirely supported by the supporting surface 53 formed by the folding portion 70 of the supporting member 50. Also, the supporting surface 53 formed by the folding portion 70 of the supporting member 50 extends in the radial direction of the lateral opening 46 with respect to the longitudinal axis over a distance which is longer than the surface actually supporting the flange 12a of the syringe 6 when said syringe 6 is loaded in the injection device. Alternatively, the folding portion 70 may not extend over the whole length of the reception space 40, and a non-folding portion of the supporting member 50 may be a complementary supporting surface that extends or prolongs the supporting surface 53 formed by the folding portion 70. As a result, when the syringe 6 is loaded into the injection device 2, the flange 12a of the syringe 6 is received in the reception space 40 and is at least partially supported by a non-folding portion of the supporting member 50, i.e. the main part 74 of the interface module 4. Also, when the folding portions 70 are in the open position, a syringe 6 whose flange 12a is in the reception space 40 and supported at least partially by the complementary supporting surface of the supporting member 50 cannot be translated along the longitudinal axis X. The syringe 6 is therefore held in the direction of the longitudinal axis X, and a traction on the syringe 6 in the longitudinal direction cannot disengage the syringe 6. Preferably, when the syringe 6 is loaded, with a flange 12a in the reception space 40, the flange 12a is also partially supported by the supporting surface 53 formed by the folding portion 70.

The interface module 4 includes also a pair of jaws 80 flanking the reception space 40, the jaws 80 being pivotable between a clamping position in which the jaws are closed in the reception space 40, and a release position in which the jaws 80 are open, i.e. are spaced apart from each other. In FIGS. 5 and 6, the jaws 80 are omitted. FIG. 7 is an exploded view of the interface module 4, showing the hinged doors 72 and the jaws 80 disassembled from the main part 74. For example, the jaws 80 are rotatably mounted on the main part 74 with a pivot 82 parallel to the longitudinal axis X. Each jaw 80 is mounted on the main part 74, one jaw 80 on either side of the reception space 40. Each jaw 80 is configured to have an inner surface 84 (on the side of the reception space 40) which is complementing the outer shape of the barrel 10 of the syringe. Preferably, the interface module 4 has a slot 50b above each groove 50a to allow the insertion of the upper flange 12b, whereas the groove 50a is meant to accommodate the lower flange 12a.

As shown in FIG. 2, the loading process for loading the syringe 6 into an injection device 2, includes radially translating the syringe 6 in a first radial direction (represented by the arrow) through the lateral opening 46 until a loaded position in which the flange 12a of the syringe 6 is accommodated in the reception space 40 and the barrel 10 of the syringe 6 extends along the longitudinal axis X.

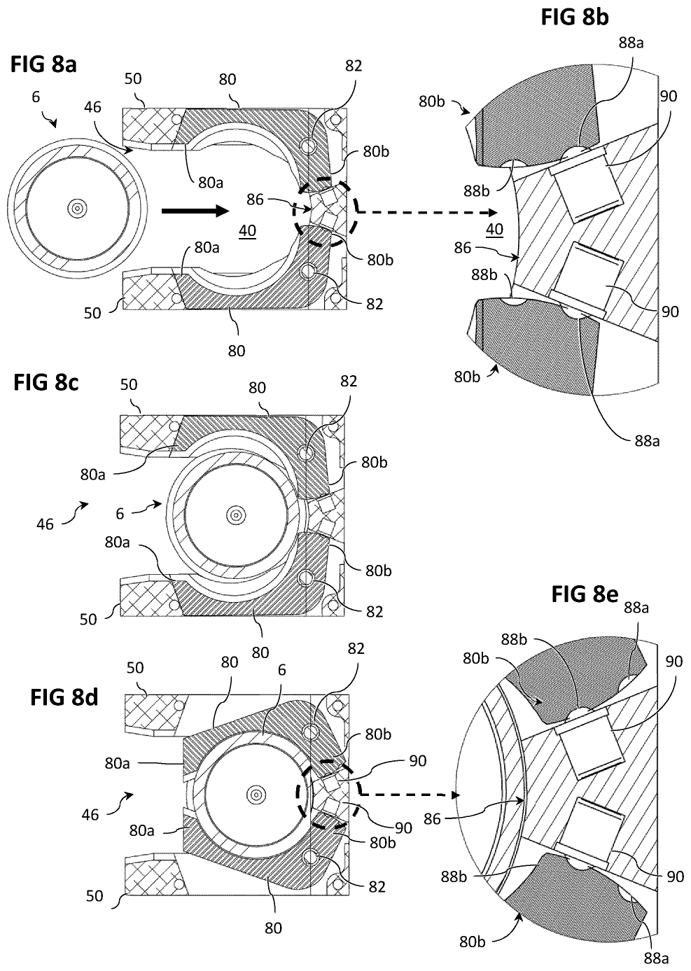

FIGS. 8a, 8b, 8c, 8d and 8e are top views in accordance with a section plane perpendicular to the longitudinal axis X which show different stages of insertion of the syringe 6 into the reception space 40 to illustrate how the jaws 80 work to grab and retain the syringe 6. The jaws 80 flank the reception space 40, the jaws 80 are pivotable between a clamping configuration in which the jaws 80 are closed in the reception space 40, and an open configuration in which the jaws are open. The jaws 80 are maintained in the clamping configuration by a biasing force, typically provided by at least one biased retainer. The jaws 80 are maintained in the open configuration by a biasing force, typically provided by at least one biased retainer. A biased retainer thus stabilizes the configuration in which the jaws are maintained. A force of enough amplitude is needed to overcome the biasing force and move the jaws from one configuration to the other. Preferably, the same biased retainer is used to stabilize the clamping configuration and the open configuration. The jaws 80 are configured to be forced out of the open configuration and into the clamping configuration by the translation of the syringe 6 into the reception space 40 in the first radial direction. The jaws 80 configured to be forced out of the clamping configuration and into the open configuration by the translation of the syringe 6 out of the reception space 40 in the second radial direction.

In a first stage illustrated by FIG. 8a, the translation of the syringe 6 toward the reception space 40 is depicted by an arrow. In FIG. 8a, the syringe 6 has reached the lateral opening 46, but not yet the reception space 40, which is therefore empty. The jaws 80 are maintained in alignment with the supporting members 50. More precisely, each jaw 80 includes a first end 80a and a second end 80b, and the pivot 82 is arranged between said first end 80a and said second end 80b. In the open configuration of the first stage, the first end 80a of each jaw 80 at the level of the lateral opening 46 is aligned with a respective supporting member 50 to allow the insertion of the syringe 6. The second end 80b protrudes partially from the back wall 86 of the main part 74 opposite the lateral opening 46, into the reception space 40.

FIG. 8b is an enlarged view of FIG. 8a showing an example of how the jaws 80 may be maintained in the open configuration by a biasing force provided by a biased retainer. The second end 80b of a jaw 80 is provided with a first recess 88a and a second recess 88b, the first recess 88a being more distant from the reception space 40 than the second recess 88b. The first and second recessed 88a, 88b of a jaw 80 face the first and second recessed 88a, 88b of the other jaw 80. For each jaw 80, a biased retainer 90 such as a ball spring, is arranged in the main part 74 of the interface module 4, and is engaged with a first recess 80a arranged in the second end 80b of the jaw. The engagement of the retainer 90 with the first recess 88a ensures that the jaw 80 cannot be moved unless a sufficient force is applied to the jaw 80.

In the second stage depicted by FIG. 8c, the syringe 6 has reached the reception space 40. By doing so, the syringe 6 presses against the protruding part of the second end 80b of the jaws 80 in the first radial direction, i.e. to the right on the figures. Consequently, the jaws 80 are subjected to a rotational force around the axis of the pivot 82. When the amplitude of this rotational force exceeds the biasing forces of the retainers 90, the retainers 90 are disengaged from the first recesses 88a, allowing the rotation of the jaws 80. The first ends 80a of the jaws 80 move into the reception space 40, closing up to each other. In the meantime, the seconds ends 80b of the jaws 80 move away from the reception space 40, allowing the syringe 6 to be received in the reception space 40. The first recesses 88a move away from the retainers 90 while the second recesses 88b get closer to the retainers 90.

The rotations of the jaws 80 continue until each second recess 88b faces the corresponding retainer 90. At that time, the biased retainer 90 engages into the corresponding second recess 88b, thus stopping the rotation of the jaw 80 and securing said jaw 80 in a clamping configuration as illustrated by FIGS. 8d and 8e. In this clamping configuration, the upper flange 12b of the syringe 6 is engaged by the inner surfaces 84 of the jaws 80, typically in a groove arranged in each inner surface 84. The upper flange 12b of the syringe 6 is thus secured by the jaws 80 while the lower flange 12a is secured by the supporting members 50. Because of the upper flange 12b of the syringe 6 cooperating with the jaws 80, the syringe 6 cannot be disengaged by a force applied in the direction of the longitudinal axis X. Only a force applied to the jaws 80 in the radial direction towards the lateral opening 46, i.e. in a so-called second radial direction, may disengage the syringe 6, provided that this force exceeds the retaining force of the retainers 90, i.e. is sufficient to disengage the retainers 90 from the second recesses 88b.

Once the syringe 6 is engaged and locked in the reception space 40, the barrel 10 of the syringe extends along the same longitudinal axis X as the translation axis of the drive member 8. The open first end 10a of the barrel 10 is also centered with regards to the cover hole 42. The drive member 8 may then translate along the longitudinal axis X to reach the reception space 40 of the interface module 4. The drive member 8 penetrates into the inner volume of the syringe 6, where is located the piston 30 of the syringe. In this example, the second coupling organ of the piston 30 is a projecting part 32, oriented toward the open first end 10a of the barrel 10, which is configured to cooperate with the first coupling organ 28 of the drive member 8 of an injection device 2 to secure the piston 30 to the drive member 8. More precisely, the drive member 8 includes a shaft 24 terminated by a head 26 which is provided with the first coupling organ 28 configured to engage and disengage the projecting part 32 of the piston 30.

FIGS. 9a, 9b and 9c show an exemplary embodiment of a cooperation between the head 26 of the drive member 8 and the projecting part 32 of the piston 30. The projecting part 32 of the piston 30 includes a head 32a and a neck 32b, in which the head 32a is larger than the neck 32b in a radial direction, and the neck 32b is connecting the head 32a to the rest of the piston 30. Preferably, the head 32a and/or the neck 32b have a circular section, and more generally, the projecting part 32 of the piston 30 is symmetrical with respect to the longitudinal axis X, so that the syringe 6 may be inserted in any angular configuration as long as the barrel 10 is parallel to the longitudinal axis X.

In one aspect of the present disclosure, the first coupling organ 28 includes a gripper 29 configured to grip the projecting part 32 of the piston 30 and to retain said projecting part 32 along the longitudinal axis X without retaining said projecting part 32 in a second, radial, direction opposite the first, radial, direction. To this end, the gripper 29 is asymmetrical with respect to the longitudinal axis, having a clearance left in said second radial direction. Preferably, and as depicted in this embodiment, the head 26 of the drive member 8 comprises a hollow end 27 which is open in the longitudinal direction, in order to allow the second coupling organ 32 to penetrate said hollow end 27, and open in the first radial direction to allow a radial translation of the second coupling organ 32 out of the hollow end 27. The hollow end 27 defines an inner volume in which the gripper 29 is arranged. The inner volume is open by a lateral opening in the second radial direction, and an open passage opposed to the shaft 24.

As in the depicted example, the gripper 29 may have claws 92 adapted to grip and hold the projecting part 32 of the piston 30. The claws 92 are pivotably mounted with a pivot axis 94 between two ends of the claws 92, in the same way as pliers. The claws 92 are biased by a biasing element 96 such as a spring into a closed configuration wherein the distance between the claws 92 is smaller than a diameter (a largest radial dimension) of the head 32a of the piston's projecting part 32. This configuration is depicted by FIG. 9a, which shows the head 26 of the drive member 8 before it contacts the projecting part 32.

As the drive member 8 penetrates further into the inner volume of the barrel 10, the head 26 of the drive member 8 presses the projecting part 32 of the piston 30. The tips of the claws 92 are designed to be spread apart when pressed against the head 32a of the projecting part 32. For example, the front surfaces of the tips of the claws 92 and the head 32a of the projecting part 32 may be complementarily chamfered or beveled in the direction of the piston 30. As shown in FIG. 9b, the action of the drive member 8 thus causes the claws 92 to open by a respective rotation around the pivot axis 94, to allow the tips of the claws 92 to pass the head 32a of the projecting part 32, which is then between the claws 92. As the drive member 8 continues moving along the longitudinal axis X, the tips of the claws 92 reach the neck 32b of the piston's projecting part 32, whose smaller section allows the claws 92 to come back in a closed configuration, as shown in FIG. 9c, under the action of the biasing element 96. Contrary their front surfaces, the back surfaces of the tips of the claws 92 and the head 32a of the projecting part 32 are not chamfered, or are chamfered in the same directions as the front surfaces. Thus, a traction of the drive member 8 will not result in the opening of the claws 92, but instead results in a blocking of the claws 92 by the head 32a.

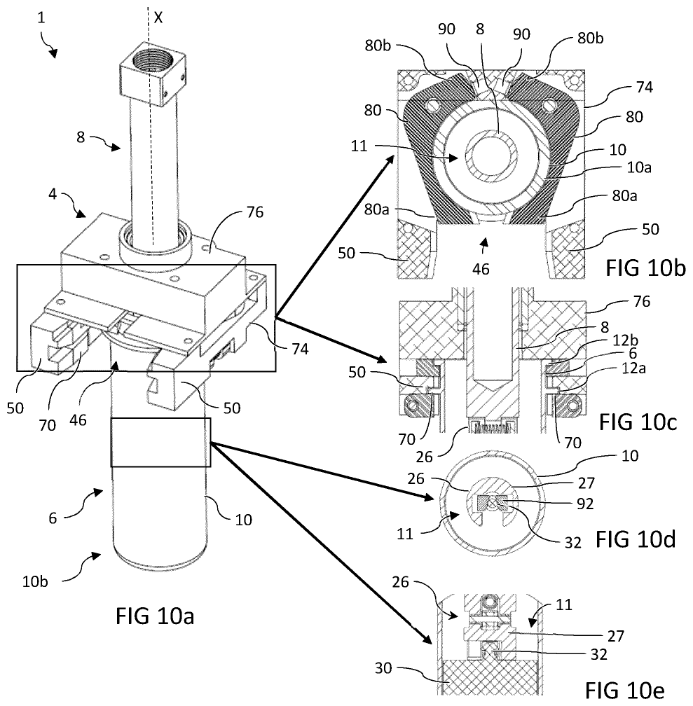

FIG. 10a shows an overview of the injection system 1 with a syringe 6 secured in the reception space 40 of the interface module 4. FIG. 10b shows a cross-section view of the injection system 1 according to a cutting plane perpendicular to the longitudinal axis X at the level of the jaws 80. FIG. 10c shows a cross-section view of the injection system 1 according to a cutting plane including the longitudinal axis X at the level of the interface module 4. FIG. 10d shows a cross-section view of the syringe 6 according to a cutting plane perpendicular to the longitudinal axis X at the level of the projecting part 32 of the piston 30. FIG. 10d shows a cross-section view of the syringe 6 according to a cutting plane including the longitudinal axis X at the level of the projecting part 32 of the piston 30.

In the loaded position illustrated by FIGS. 10a-10e, the flange 12a of the syringe 6 is received in the reception space 40 and the barrel 10 of the syringe 6 extends along the longitudinal axis X, in alignment with the direction of translation of the drive member 8. The lower flange 12a is maintained in the longitudinal direction by the supporting surfaces 53 of the folding portions of the supporting members 50. The upper flange 12b is clamped by the jaws 3, thereby securing the syringe 6 to the interface module 4 in at least the radial direction, and preferably also in the longitudinal direction. The drive member 8 and the piston 30 are coupled by means of their respective coupling organs (28, 32). The injection system 1 may now be operated to pump and inject a medical fluid. The drive member 8 translates along the longitudinal axis X and causes the piston 30 to similarly translate along the longitudinal axis X thereby causing a vacuum or overpressure in the portion of the barrel 10 defined between said piston 30 and the second end 10b of the barrel which is connected to the tubing. The medical fluid may then be sucked into the barrel 10 or pressurized out of the barrel 10.

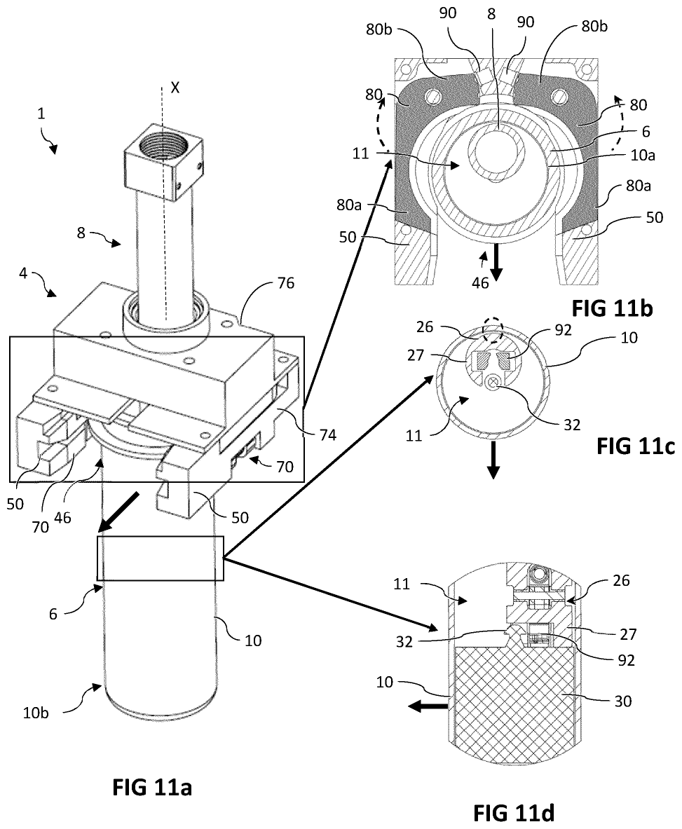

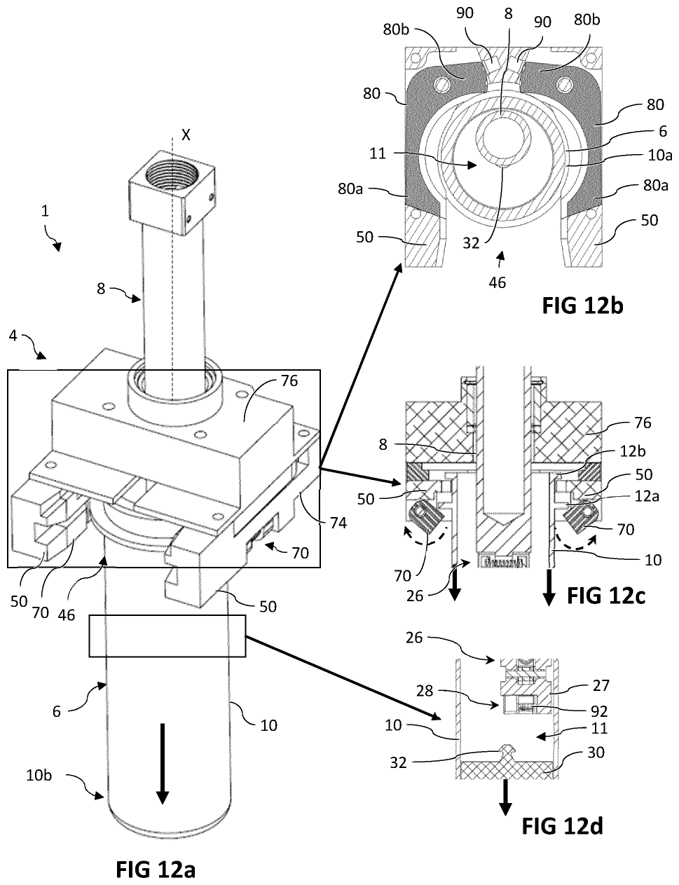

With reference to FIGS. 11a to 12c, a process for unloading the syringe 6 from the injection device 2 will now be described. The removal of the syringe 6 includes two steps:
a first step of radially translating the syringe 6 in a second radial direction, opposite the first radial direction, toward the lateral opening 46 of the interface module 4, so that a flange 12 of said syringe faces folding portions 70 of the supporting members 50, and
a second step of longitudinally translating the syringe 6 in a third, longitudinal, direction parallel to the longitudinal axis, thereby causing the folding portions 70 of the supporting members 50 to fold and allow a longitudinal translation of the syringe 6 out of the loaded position.

FIGS. 11a to 11d illustrate the first step of the removal of the syringe 6. As noted by an arrow in FIG. 11a, a force is applied to the syringe 6 in the radial direction towards the lateral opening 46, i.e. in a second radial direction opposed to the first radial direction. The jaws 80 maintaining the syringe 6 are thus subjected to a rotational force tending to open the jaws 80. When the amplitude of this rotational force exceeds the biasing forces of the retainers 90, the retainers 90 are disengaged from the second recesses 88b, allowing the rotation of the jaws 80 into the open configuration. The first ends 80a of the jaws 80 move out of the reception space 40, moving away from each other. In the meantime, the seconds ends 80b of the jaws 80 penetrate the reception space 40. The second recesses 88b move away from the retainers while the first recesses 88a get closer to the retainers 90. The rotations of the jaws 80 continue until each first recess 88a faces the corresponding retainer 90. At that time, each biased retainer 90 engages into the corresponding first recess 88a, thus stopping the rotation of the jaw 80 and securing said jaw 80 in an open configuration as illustrated by FIG. 11b and FIG. 8a.

The opening of the jaws 80 allows the radial translation of the syringe 6 toward the lateral opening 46. More precisely, the syringe 6 is radially shifted, because the drive member 8 is still engaged in the inner volume 11 of the barrel 10, and therefore limits the amplitude of the syringe's radial translation. As illustrated by FIG. 11c, a contact may occur between the inner wall of the barrel 10 and a part of the drive member 8 such as the shaft 24, thereby effectively stopping the radial translation of the syringe 6. The amplitude of the radial translation of the syringe involved in the first step is thus defined by the difference between the outer section of the drive member's shaft 24 and the inner section of the barrel 10, in a plane perpendicular to the longitudinal axis X. There are other possibilities for stopping or defining amplitude of the radial translation of the syringe 6, but using the contact between the drive member's shaft 24 and of the inner section of the barrel 10 is particularly advantageous in that the motion is automatically stopped in the intended position, without any risk of error and without requiring any skill or knowledge from the operator.

Preferably, due to the offset of the folding portion 70 of the supporting member 50 with respect to the longitudinal axis X, the supporting surface 53 formed by the folding portion 70 extends beyond the reception space 40 towards the lateral opening 46 at least over a distance corresponding to the amplitude of the radial translation of the syringe allowed by the drive member 8 within the syringe 6. Accordingly, the movement of the syringe 6 is stopped when the part of the flange 12a of the syringe 6 supported by the supporting members 50 still faces the folding portions 70 of said supporting members 50.

The piston 30 of the syringe 6 is also radially shifted with respect to the longitudinal axis X. As shown in FIGS. 11c and 11d, the gripper 29 engages the projecting part 32 on either side of the radial direction of the lateral opening 46, but a clearance is left in said radial direction. As a consequence of the radial translation of the projecting part 32, the projecting part 32 of the piston 30 is disengaged from the gripper 29 due to the gripper 29 not restraining the projecting part 32 in this direction. The piston 30 is thus released from the drive member 8 in a completely passive way, without requiring any action such as an opening of claws 92.

At the end of the first step, the syringe 6 is still axially retained by the supporting members 50. More precisely, a flange 12a of the syringe is supported by the folding portion 70 of the supporting member 50.

FIGS. 12a to 12c illustrate the second step of the syringe's removal. In this second step, a longitudinal force, parallel to the longitudinal axis (downwards in the FIG. 12a as shown by the arrow), is applied to the syringe 6. When the applied force exceeds a biasing force keeping the folding portions 70 closed (for example provided by the torsion spring), the folding portions 70 of the supporting members 50 fold and allow a longitudinal translation of the syringe 6 out of the reception space 40. More precisely, with reference to the depicted example, each hinged door 72 pivots perpendicularly with respect to the longitudinal axis X from its support position to the release position, as indicated by arrows in dotted lines on FIG. 12b. The drive member 8 still extends within the syringe's barrel 10. Since the syringe's piston 30 was previously disengaged from the drive member 8, and because the syringe is translated in a third direction parallel to the longitudinal axis X along which extends the drive member 8, the drive member 8 does not prevent the syringe's translation. The drive member 8 may guide the longitudinal translation as long as the drive member 8 extends within the barrel 10.

The longitudinal translation of the syringe 6 is continued until the drive member 8 no longer extends within the barrel 10. At that point, the syringe 6 is removed. It shall be noted that the removal of the syringe 6 does not require any knowledge or control of the position of the drive member. In order to insert a new syringe 6 into the injection device, it is however necessary to retract the drive member 8 so that it no longer passes through the reception space 40.

The embodiments described above, it is required that each supporting member 50 comprises a folding portion 70 pivotable between a supporting position in which said folding portion 70 forms a supporting surface 53 for the flange 12a of the syringe 6, and an open position in which said folding portion 70 opens a passage parallel to the longitudinal axis X for the flange 12a of the syringe 6, thereby allowing a longitudinal translation of the syringe 6 out of the loaded position. The described longitudinal translation of the syringe 6 out of the loaded position allows removing the syringe 6 regardless of the position of the drive member 8 in the inner volume 11 of the syringe.

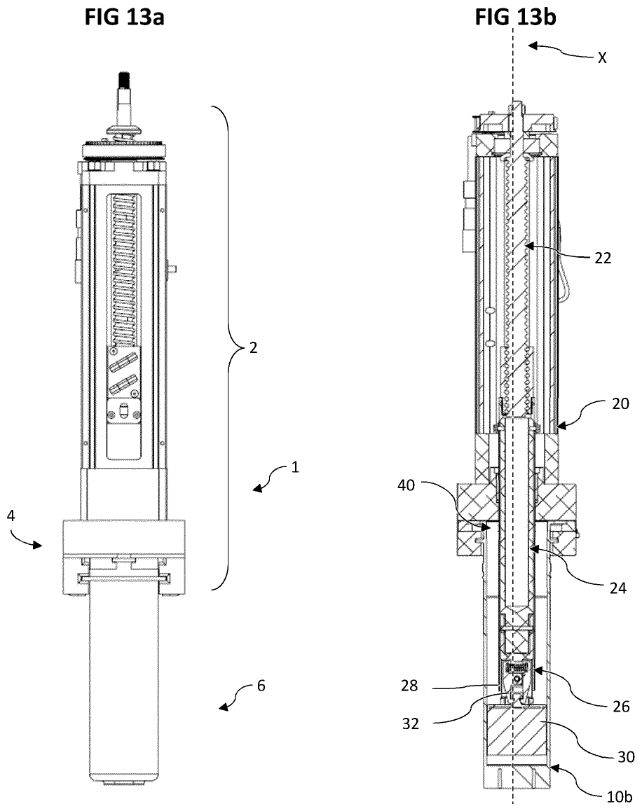

Other embodiments do not have such a requirement, but the position of the drive member 8 in the inner volume 11 of the syringe can no longer be disregarded. FIGS. 13a to 17d disclose an example of such an embodiment, wherein the interface module 4 is not provided with a folding portion 70, or at least the folding portion 70 is not used in the unloading process. It shall be noted that every feature disclosed in relation with the embodiments above can also be found in the embodiments described below.

FIG. 13a shows a view of an injection system 1, and FIG. 13b shows a cross-section of the injection system 1. The injection system 1 includes an injection device 2 including an interface module 4 in which is loaded a syringe 6, in the mounted configuration wherein the injection system 1 can be used. Such an injection system 1 is used to inject any medical fluid into a patient, such as contrast agent. The medical fluid is provided from a medical fluid container (not shown) coupled to the syringe 6. Typically, the injection device 2 is sized and configured to accommodate use for multiple injections (e.g., multiple injections of one, but more typically multiple patients) and with different medical fluids, whereas the syringe 6 is configured to be a single-use device, for example for one type of medical fluid or for one patient only, one for a limited duration of time (daily set). The injection device 2 includes a drive member 8 configured to translate along a longitudinal axis, thereby defining said longitudinal axis. A radial direction is a direction extending in a plane perpendicular to the longitudinal axis and intersecting said longitudinal axis.

In most of the figures such as FIGS. 13a and 13b, the injection system 1 is represented in a vertical configuration, i.e. with the longitudinal axis aligned with the vertical, with the injection device 2 above the syringe 6. There is however no such positioning requirement, and the injection system could be rotated in an oblique or horizontal configuration. However, for the sake of simplicity and clarity, the following description may refer to relative spatial indications such as “upwards”, “downwards”, “above”, “below”, etc., which must be construed as non-limiting and used to describe the appended drawings.

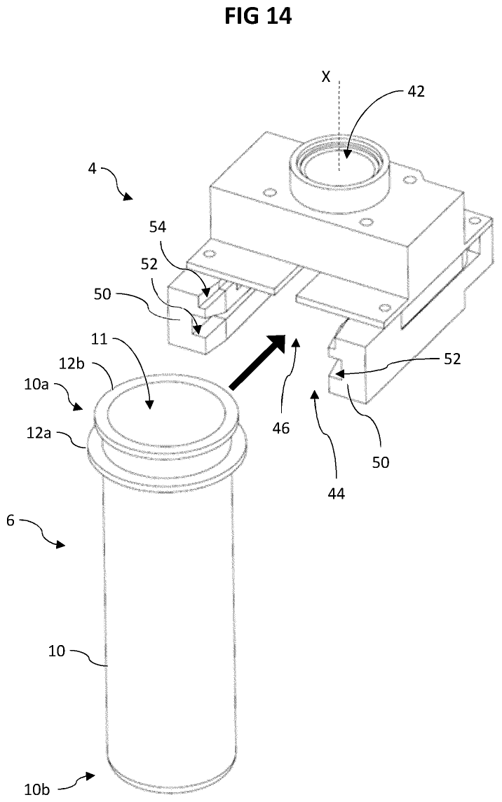

FIG. 14 more clearly shows the syringe 6 and the interface module 4. The syringe 6 includes a barrel 10, preferably a cylindrical hollow body, with a first end 10a and a second end 10b, which defines an inner volume 11 for the syringe 6. The inner volume 11 extends between the first end 10a and the second end 10b. The first end 10a is open and allows a drive member 8 of the injection device 2 to translate along the longitudinal axis within the barrel 10 of the syringe 6. The second end 10b includes a tubing interface adapted for connecting a tubing to the syringe 6 for transporting the medical fluid into and out of the syringe 6. The syringe 6 is provided with a piston 30 located within the inner volume 11 defined by the barrel 10. Preferably, the syringe 6 is symmetrical with respect to the longitudinal direction.

The barrel 10 is provided with at least a flange 12a in the outer periphery of the barrel 10. The flange 12a continuously encircles the barrel 10, and is perpendicular to the longitudinal axis X. The flange 12a has a circular shape. Preferably, as depicted in the figures, the barrel 10 is provided with two flanges 12a, 12b: an upper flange 12b and a lower flange 12a, the upper flange 12b closer to the open first end 10a of the barrel 10 than the lower flange 12a. Both flanges 12a, 12b are however close to each other, at the same side of the barrel 5 near the first end 10a and at distance from the second end 10b. The flanges are preferably similar in shape. A flange may form the extremity of the barrel 10 at the first end 10a thereof, as the upper flange 12b in the depicted example. In one aspect of the present disclosure, the lower flange 12a has a larger outer diameter than the upper flange 12b to facilitate downward extraction.

The injection device 2 includes an interface module 4 configured to receive the syringe 6, and a drive member 8 (illustrated in FIGS. 9a-9c). The drive member 8 is configured to translate along a longitudinal axis through the interface module 4 and within the barrel 10 of the syringe 6. The injection device comprises an actuator 20 for driving the drive member 8 into translation. The actuator 20 is typically a linear actuator, and most notably an electric actuator including an electric motor mechanically connected to rotate a lead screw 22 on which is mounted a mobile shaft 24 which is translated along the longitudinal axis X, and forms the part of the drive member 8 configured to traverse the interface module to translate within the barrel 10. Other types of actuators may be used. The drive member 8 is configured to couple with a piston 30 of the syringe 6. More precisely, the drive member 8 is terminated by a head 26 provided with a first coupling organ 28 configured to couple to a second coupling organ 32 of the syringe's piston 30 when said first coupling organ 28 is pressed on said second coupling organ 32.

The interface module 4 forms an interface between the drive member 8 and the syringe 6. The interface module 4 defines a reception space 40 adapted to receive and accommodate the flange 12b of a syringe 6, with a syringe's barrel 10 extending along the longitudinal axis X. In FIGS. 13a and 13b, the syringe 6 is loaded in the injection device 2 and therefore the first end 10a of the barrel 10 is received in the reception space 40 of the interface module 4 of the injection device 2. More precisely, the two flanges 12a, 12b are inserted into the interface module 4 and are accommodated in the reception space 40.

The interface module 4 also includes a hole 42 along the longitudinal axis X to allow the drive member 8 to penetrate into the reception space 40. In the depicted example, the through hole is provided with connection means such as a thread so that a housing of the actuator 20 can be fixed to the interface module 4. The interface module 4 has an open side 44 opposite the hole 42, in communication with the reception space 40, to allow the syringe 6 to extend along the longitudinal axis X when the syringe 6 is loaded in the first interface module. The interface module 4 includes a lateral opening 46 in communication with the reception space 40 and adapted for a radial translation of the syringe 66 in a first, radial, direction to a loaded position in which the flange 12a of the syringe 6 is received in the reception space 40 and the barrel 10 of the syringe 6 extends along the longitudinal axis X, thanks to the open side 44 of the interface module 4. The lateral opening 46 thus extends in a radial direction from the longitudinal axis X, so that the reception space 40 is laterally opened by said lateral opening 46.

The lateral opening 46 is defined between two supporting members 50. Each supporting member 50 has a guiding surface 52 extending in the radial direction to guide the flange 12a of the syringe during the insertion of the syringe. Preferably, as shown, each supporting member 50 has a guiding surface 52, 54 for each of the two flanges 12a, 12b: a lower guiding surface 52 for the lower flange 12a and an upper guiding surface 54 for the upper flange 12b. As shown in this example, a guiding surface 52 can be formed by a groove or slit arranged in a supporting member 50. Each supporting member 50 also has a supporting surface 53 extending in the radial direction to maintain and support a flange 12a in the longitudinal direction. The supporting surface 53 extends or prolongs the guiding surface 53 in the reception space 40.

FIG. 14 shows a single syringe 6 about to be loaded into an interface module 4 with a single lateral opening 46 and a single reception space 40. A fat arrow shows the first radial direction for the radial translation of the syringe 6 required for loading syringe 6 in the interface module 4, until a loaded position in which the flange 12a of the syringe 6 is accommodated in the reception space 40 and the barrel 10 of the syringe 6 extends along the longitudinal axis. As explained above with respect to FIGS. 3 and 4, several syringes may be inserted in an interface module. Especially, it is possible to use a syringe assembly comprising at least one syringe 6 mounted on a holder 56, wherein each syringe 6 comprising comprises a barrel 10 extending along a longitudinal direction and defining an inner volume 11 extending between a first end 10a and a second end 10b, wherein the first end 10a is open and allows a drive member 8 of an injection device 2 to translate along a longitudinal axis within the barrel 10, the each syringe 6 comprising a piston 30 within the barrel 10, said piston head comprising a coupling organ 32 configured to cooperate with the drive member 8 of an injection device, wherein the barrel 10 is provided with at least a flange 12a in the outer periphery of the barrel 10.

FIG. 15 shows details of the interface module 4 in an exemplary embodiment. The interface module 4 includes a main part 74 on which is fixed a cover 76. The cover hole 42 goes through the cover 76 and ensures the centering and guidance of the drive member 8 passing through with respect to the interface module 4. The cover hole therefore preferably has a section corresponding to the section of the drive member. The cover 76 may be fixed on the main part 74 by fastening organs such as screws passing through fixation holes provided in the cover 76 and main part 74.

The main part 74 of the interface module has one supporting member 50, or arm, on either side of the lateral opening 46. The two supporting members 50 define two respective guiding surfaces 52 and non-folding supporting surfaces 53 for a syringe's flange 12a. When the syringe 6 is loaded into the injection device 2, the flange 12a of the syringe 6 is received in the reception space 40 and is supported by a non-folding supporting surface 53 of the supporting member 50, i.e. the main part 74 of the interface module 4. The syringe 6 is therefore held in the direction of the longitudinal axis X, and a traction on the syringe 6 in the longitudinal direction cannot disengage the syringe 6.

The interface module 4 includes also a pair of jaws 80 flanking the reception space 40, the jaws 80 being pivotable between a clamping position in which the jaws are closed in the reception space 40, and a release position in which the jaws 80 are open, i.e. are spaced apart from each other. For example, the jaws 80 are rotatably mounted on the main part 74 with a pivot 82 parallel to the longitudinal axis. Each jaw 80 is mounted on the main part 74, one jaw 80 on either side of the reception space 40. Each jaw 80 is configured to have an inner surface 84 (on the side of the reception space 40) which is complementing the outer shape of the barrel 10 of the syringe.

As shown in FIG. 14, the loading process for loading the syringe 6 into an injection device 2, includes radially translating the syringe 6 in a first radial direction (represented by the arrow) through the lateral opening 46 until a loaded position in which the flange 12a of the syringe 6 is accommodated in the reception space 40 and the barrel 10 of the syringe 6 extends along the longitudinal axis X. The loading process is described above in relation with FIGS. 8a, 8b, 8c, 8d and 8e which show different stages of insertion of the syringe 6 into the reception space 40. Similarly, the engagement of the coupling organs 28, 32 is described above in relation with FIGS. 9a, 9b, and 9c.

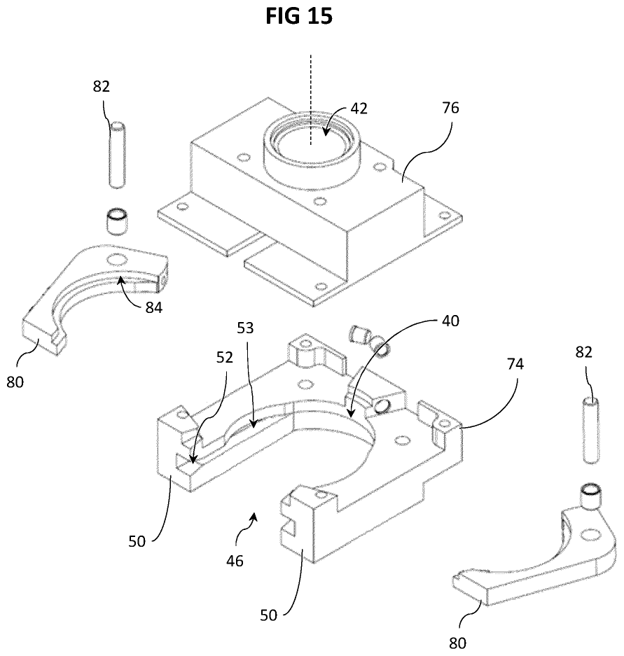

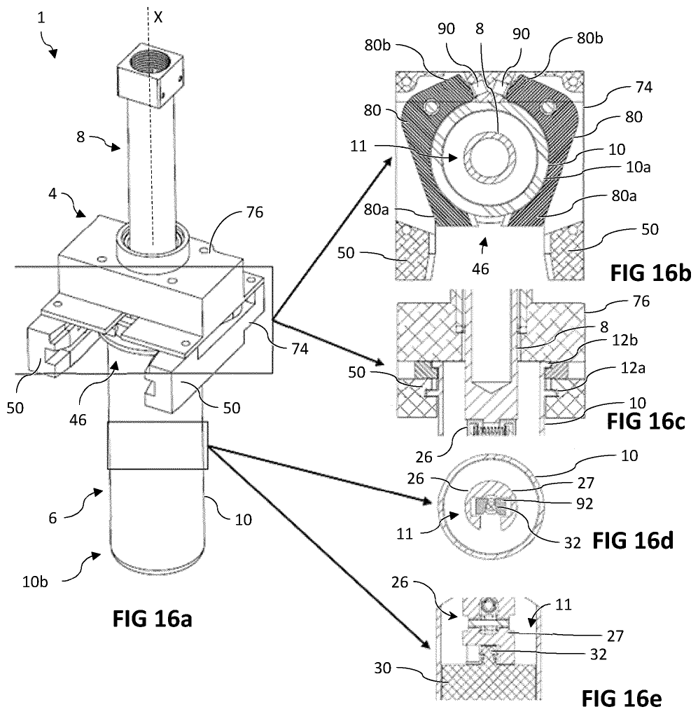

FIG. 16a shows an overview of the injection system 1 with a syringe 6 secured in the reception space 40 of the interface module 4. FIG. 16b shows a cross-section view of the injection system 1 according to a cutting plane perpendicular to the longitudinal axis X at the level of the jaws 80. FIG. 16c shows a cross-section view of the injection system 1 according to a cutting plane including the longitudinal axis X at the level of the interface module 4. FIG. 16d shows a cross-section view of the syringe 6 according to a cutting plane perpendicular to the longitudinal axis X at the level of the projecting part 32 of the piston 30. FIG. 16d shows a cross-section view of the syringe 6 according to a cutting plane including the longitudinal axis X at the level of the projecting part 32 of the piston 30.

In the loaded position illustrated by FIGS. 16a-16e, the flange 12a of the syringe 6 is received in the reception space 40 and the barrel 10 of the syringe 6 extends along the longitudinal axis X, in alignment with the direction of translation of the drive member 8. The lower flange 12a is maintained in the longitudinal direction by the non-folding supporting surfaces 53 of the supporting members 50. The upper flange 12b is clamped by the jaws 3, thereby securing the syringe 6 to the interface module 4 in at least the radial direction, and preferably also in the longitudinal direction. The drive member 8 and the piston 30 are coupled by means of their respective coupling organs 28, 32. The injection system 1 may now be operated to pump and inject a medical fluid. The drive member 8 translates along the longitudinal axis X and causes the piston 30 to similarly translate along the longitudinal axis X thereby causing a vacuum or overpressure in the portion of the barrel 10 defined between said piston 30 and the second end 10b of the barrel which is connected to the tubing. The medical fluid may then be sucked into the barrel 10 or pressurized out of the barrel 10.

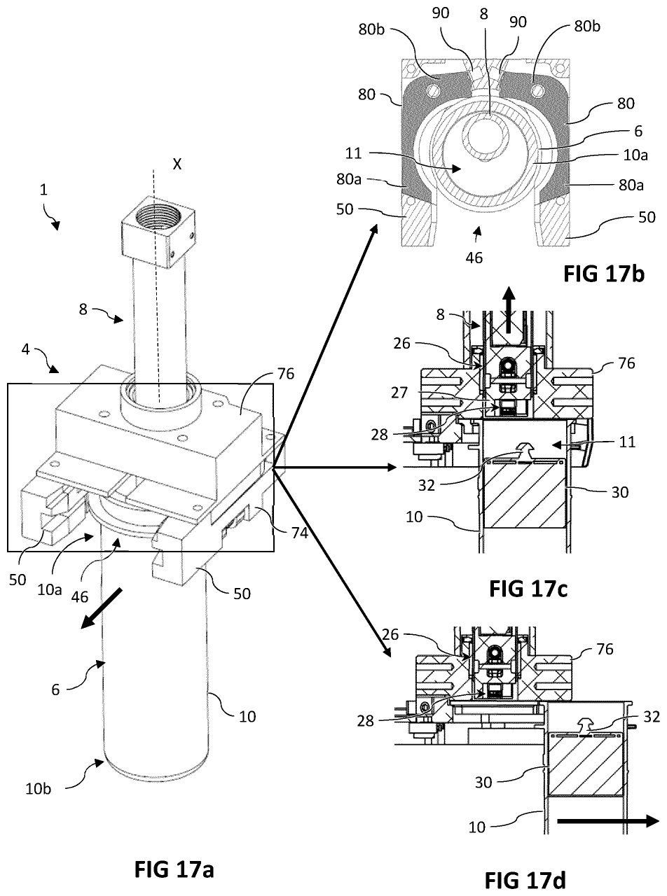

With reference to FIGS. 17a to 17d, a process for unloading the syringe 6 from the injection device 2 without using the folding portion 70 will now be described. The removal of the syringe 6 includes three steps:
a first step of radially translating the syringe in a second radial direction, opposite the first radial direction, from the reception space 40 toward the lateral opening 46 of the interface module 4, thereby disengaging the first coupling organ 28 and the second coupling organ 32,
a second step of removing the drive member 8 from an inner volume 11 of the syringe 6,
a third step of radially translating the syringe 6 in the second radial direction out of the lateral opening 46 of the interface module 4.

As noted by an arrow in FIG. 17a, a force is applied to the syringe 6 in the radial direction towards the lateral opening 46, i.e. in a second radial direction opposed to the first radial direction. The jaws 80 maintaining the syringe 6 are thus subjected to a rotational force tending to open the jaws 80. When the amplitude of this rotational force exceeds the biasing forces of the retainers 90, the retainers 90 are disengaged from the second recesses 88b, allowing the rotation of the jaws 80 into the open configuration. The first ends 80a of the jaws 80 move out of the reception space 40, moving away from each other. In the meantime, the seconds ends 80b of the jaws 80 penetrate the reception space 40. The second recesses 88b move away from the retainers while the first recesses 88a get closer to the retainers 90. The rotations of the jaws 80 continue until each first recess 88a faces the corresponding retainer 90. At that time, each biased retainer 90 engages into the corresponding first recess 88a, thus stopping the rotation of the jaw 80 and securing said jaw 80 in an open configuration as illustrated by FIG. 8a.

The opening of the jaws 80 allows the radial translation of the syringe 6 toward the lateral opening 46. More precisely, the syringe 6 is radially shifted, because the drive member 8 is still engaged in the inner volume 11 of the barrel 10, and therefore limits the amplitude of the syringe's radial translation. As illustrated by FIG. 17b, a contact may occur between the inner wall of the barrel 10 and a part of the drive member 8 such as the shaft 24, thereby effectively stopping the radial translation of the syringe 6. The amplitude of the radial translation of the syringe involved in the first step is thus defined by the difference between the outer section of the drive member's shaft 24 and the inner section of the barrel 10, in a plane perpendicular to the longitudinal axis X. There are other possibilities for stopping or defining amplitude of the radial translation of the syringe 6, but using the contact between the drive member's shaft 24 and of the inner section of the barrel 10 is particularly advantageous in that the motion is automatically stopped in the intended position, without any risk of error and without requiring any skill or knowledge from the operator.

The first coupling organ 28 is configured not to retain the second coupling organ (i.e. the projecting part 32) in a second radial direction opposite the first radial direction. As a consequence of the radial translation of the syringe 6, the second coupling organ 32 is disengaged from the first coupling organ 28. In this example, the projecting part 32 of the piston 30 of the syringe 6 is radially shifted with respect to the longitudinal axis X. As a consequence of the radial translation of the projecting part 32, the projecting part 32 of the piston 30 is disengaged from the gripper 29 due to the gripper 29 not restraining the projecting part 32 in this direction. The piston 30 is thus released from the drive member 8 in a completely passive way, without requiring any action from the second coupling organ (e.g. the projecting part 32) such as an opening of claws 92.

At the end of the first step, the syringe 6 is still axially retained by the supporting members 50. More precisely, a flange 12a of the syringe is supported by the non-folding supporting surface 53 of the supporting member 50. However, the drive member 8 and the piston 30 are no longer coupled, and the drive member 8 is free to translate without pulling the piston 30.

In a second step illustrated by FIG. 17c, the drive member 8 is removed from the inner volume 11 of the syringe 6. The drive member 8 is retracted until the head 26 of the drive member is out of the syringe 6, beyond the upper end 10a of the barrel 10. The drive member 8 thus no longer restrict the radial translation of the syringe 6 in the second radial direction, thereby allowing the third step. In this third step illustrated by FIG. 17d, the syringe 6 is translated in the second radial direction out of the lateral opening of the interface module 4.

It shall be noted that this unloading process can be performed with an injection device 2 whose supporting members 50 each comprise a folding portion 70. In this case however, during the unloading process, the folding portions 70 are kept in the supporting position in which said folding portions 70 form supporting surfaces 53 for the flange 12a of the syringe 6. Indeed, no longitudinal force is applied for pivoting said folding portions 70 into the open position in which said folding portions 70 open a passage parallel to the longitudinal axis for the flange 12a of the syringe 6.

While the present invention has been described with respect to certain preferred embodiments, it is obvious that it is in no way limited thereto and it includes all the technical equivalents of the means described and their combinations. In particular, it will be apparent to those skilled in the art that various changes and modifications may be made without departing from the scope of the invention as defined in the appended claims.

1. A injection device for delivering a medical fluid, comprising:
an interface module configured to receive a syringe, the interface module defining a reception space adapted to receive a flange in the outer periphery of a barrel of the syringe,
a drive member configured to couple a piston of the syringe, wherein said drive member is configured to translate along a longitudinal axis through the reception space,
wherein the interface module comprises a lateral opening defined between two supporting members of the interface module and extending radially in a first radial direction from the longitudinal axis, said lateral opening being adapted for a radial translation of the syringe in the first radial direction to a loaded position in which the flange of the syringe is received in the reception space and the barrel of the syringe extends along the longitudinal axis, and
wherein each supporting member comprises a folding portion pivotable between a supporting position in which said folding portion forms a supporting surface for the flange of the syringe, and an open position in which said folding portion opens a passage parallel to the longitudinal axis for the flange of the syringe, thereby allowing a longitudinal translation of the syringe out of the loaded position.

2. The injection device of claim 1, wherein the folding portion of the supporting member is radially offset with respect to the longitudinal axis, in the radial direction of the lateral opening.

3. The injection device of claim 2, wherein the supporting surface formed by the folding portion extends in a second radial direction, opposite the first radial direction, beyond the reception space towards the lateral opening.

4. The injection device of claim 1, wherein the folding portion of a supporting member is formed by a hinged door pivotable perpendicularly with respect to the longitudinal axis between the supporting position, and the open position.

5. The injection device of claim 4, wherein the hinged door is biased into the supporting position by a biasing member.

6. The injection device of claim 1, wherein the drive member comprises a head provided with a first coupling organ configured to couple to a second coupling organ of the piston when said first coupling organ is pressed on said second coupling organ, wherein the first coupling organ is configured to retain said second coupling organ along the longitudinal axis without retaining said second coupling organ in a second radial direction opposite the first radial direction.

7. An injection device for delivering a medical fluid, comprising:
an interface module configured to receive a syringe, the interface module defining a reception space adapted to receive a flange in the outer periphery of a barrel of the syringe,
a drive member configured to couple a piston of the syringe, wherein said drive member is configured to translate along a longitudinal axis through the reception space,
wherein the interface module comprises a lateral opening defined between two supporting members of the interface module and extending radially in a first radial direction from the longitudinal axis, said lateral opening being adapted for a radial translation of the syringe in the first radial direction to a loaded position in which the flange of the syringe is received in the reception space and the barrel of the syringe extends along the longitudinal axis, and
wherein the drive member comprises a head provided with a first coupling organ configured to couple to a second coupling organ of the piston when said first coupling organ is pressed on said second coupling organ as the drive member translates along the longitudinal axis into the inner volume of the barrel, wherein the first coupling organ is configured to retain said second coupling organ along the longitudinal axis without retaining said second coupling organ in a second radial direction opposite the first radial direction.

8. The injection device of claim 1, wherein the interface module comprises a pair of jaws flanking the reception space, the jaws being pivotable between a clamping position in which the jaws are closed in the reception space, and an open configuration in which the jaws are open, wherein said pair of jaws are maintained in the clamping configuration by a biasing force, and wherein said pair of jaws are maintained in the open configuration by a biasing force.

9. A syringe assembly comprising a plurality of syringes mounted on a same holder, wherein each syringe comprises a barrel extending along a longitudinal direction and defining an inner volume extending between a first end and a second end, wherein the first end is open and allows a drive member of an injection device to translate along a longitudinal axis within the barrel, each syringe comprising a piston within the barrel, wherein the barrel is provided with at least a flange in the outer periphery of the barrel,
wherein the piston comprises a coupling organ configured to cooperate with the drive member of an injection device, said coupling organ being a projecting part comprising a head and a neck, said head larger than the neck in a radial direction, said neck connecting the head to the rest of the piston.

10. The syringe assembly of claim 9, wherein the holder is provided with a plurality of brackets for holding a respective syringe, wherein the syringes are aligned.

11. The syringe assembly of claim 9, wherein the barrel of each syringe is provided with at least a flange in the outer periphery of the barrel, said flange being configured to be received in a reception space of an interface module of an injection device in accordance with claim 1 in a configuration in which the barrel extends along the longitudinal axis.

12. The syringe assembly of claim 9, wherein the barrel of each syringe is provided with an upper flange and a lower flange in the outer periphery of the barrel, said two flanges distributed along the longitudinal direction, the upper flange closer to the open first end of the barrel than the lower flange.

13. An injection system comprising the injection device of claim 1 and the syringe assembly of claim 9.

14. An unloading process for removing a syringe from an injection device in an injection system of claim 13, wherein each supporting member comprises a folding portion pivotable between a supporting position in which said folding portion forms a supporting surface for the flange of the syringe, and an open position in which said folding portion opens a passage parallel to the longitudinal axis for the flange of the syringe, thereby allowing a longitudinal translation of the syringe out of the loaded position, said unloading process comprising two steps:
a first step of radially translating the syringe in a second radial direction, opposite the first radial direction, from the reception space toward the lateral opening of the interface module, so that a flange of said syringe faces a folding portion of a supporting member, and
a second step of longitudinally translating the syringe in a third, longitudinal, direction parallel to the longitudinal axis, thereby causing the folding portion of the supporting member to fold and allow a longitudinal translation of the syringe out of the loaded position.

15. An unloading process for removing a syringe from an injection device in an injection system of claim 13, wherein the drive member comprises a head provided with a first coupling organ configured to couple to a second coupling organ of the piston when said first coupling organ is pressed on said second coupling organ, wherein the first coupling organ is configured to retain said second coupling organ along the longitudinal axis without retaining said second coupling organ in a second radial direction opposite the first radial direction, said unloading process comprising three steps:
a first step of radially translating the syringe in a second radial direction, opposite the first radial direction, from the reception space toward the lateral opening of the interface module, thereby disengaging the first coupling organ and the second coupling organ,
a second step of removing the drive member from an inner volume of the syringe,
a third step of radially translating the syringe in the second radial direction out of the lateral opening of the interface module.

16. The injection device of claim 7, wherein the interface module comprises a pair of jaws flanking the reception space, the jaws being pivotable between a clamping position in which the jaws are closed in the reception space, and an open configuration in which the jaws are open, wherein said pair of jaws are maintained in the clamping configuration by a biasing force, and wherein said pair of jaws are maintained in the open configuration by a biasing force.

17. An injection system comprising the injection device of claim 7 and the syringe assembly of claim 9.

18. The syringe assembly of claim 9, wherein the barrel of each syringe is provided with at least a flange in the outer periphery of the barrel, said flange being configured to be received in a reception space of an interface module of an injection device in accordance with claim 7 in a configuration in which the barrel extends along the longitudinal axis.
